(19) United States
(12) Patent Application Publication
(10) Pub. No.: US 2009/0062679 A1
(43) Pub. Date: Mar. 5, 2009

(54) CATEGORIZING PERCEPTUAL STIMULI BY DETECTING SUBCONCIOUS RESPONSES

(75) Inventors: Desney Tan, Kirkland, WA (US); Pradeep Shenoy, Seattle, WA (US)

Correspondence Address: MICROSOFT CORPORATION;C/O LYON & HARR, LLP, 300 ESPLANADE DRIVE, SUITE 800, OXNARD, CA, 93036, US

(73) Assignee: Microsoft Corporation, Redmond, WA (US)

(21) Appl. No.: 11/845,583

(22) Filed: Aug. 27, 2007

(51) Int. Cl. A61B 5/0484

(52) U.S. Cl. 600/544

(57) ABSTRACT

A perceptual stimulus categorization technique is presented which identifies the stimuli category of a perceptual stimulus that has been presented to a person whose brain activity is being monitored. This generally accomplished by first training a detection module to recognize the part of the brain activity generated in response to the presentation of a stimulus belonging to each of one or more stimuli categories using brain activity information. Once the detection module is trained, a subsequent instance of a stimulus belonging to a trained stimuli category being presented to the person is detected, and this detection is used to identify the trained stimuli category to which the presented stimulus belongs.

### BACKGROUND

The human brain implicitly processes a large amount of environmental information that a person may never become aware of. In fact, humans cannot help but process this information, even when they are actively trying not to. This occurs because awareness of a stimulus is generally thought to be preceded by subconscious information processing. The physical features of verbal or visual stimuli are subconsciously analyzed within the first 250 ms or so after being presented. While the subconscious processing system is able to simultaneously analyze the physical properties of multiple stimuli, the channel used for conscious analysis of a stimulus has limited parallel processing capacity. Consequently, only some of the subconsciously processed information can be selected for conscious processing. Thus, the subconscious processes stimuli that we may never even become aware of and which remain in the attentional periphery of a person's mind.

### SUMMARY

This Summary is provided to introduce a selection of concepts, in a simplified form, that are further described below in the Detailed Description. The Summary is not intended to identify key features or essential features of the claimed subject matter, nor is it intended to be used as an aid in determining the scope of the claimed subject matter.

Recent advances in cognitive neuroscience and brain sensing technologies provide means to interface relatively directly with activity in the brain and measure some of the presence and output of this processing. In many cases, the aforementioned subconscious processing of stimuli can be detected. Thus, new opportunities exist to harness the power of the brain to perform useful tasks, such as categorizing perceptual stimuli being presented to a person, even when that person is not aware of the task and not trying to perform it.

In general, the present perceptual stimulus categorization technique entails identifying the category of a perceptual stimulus that has been presented to a person whose brain activity is being monitored. In one embodiment of the technique, this first involves training a detection module to recognize (in a representative signal) brain activity generated in response to the presentation of a stimulus belonging to each of one or more categories of perceptual stimuli. Once the detection module is trained, a subsequent instance of a stimulus presented to the person is detected and the stimuli category that the stimulus belongs to is identified. The stimulus is then designated as belonging to the identified category.

The detection module training involves first presenting stimuli belonging to each of one or more categories of perceptual stimuli of interest that are later to be used to identify the category of a stimulus presented to a monitored person. To this end, the signals from a brain activity sensing device used to monitor the person are input. These signals exhibit distinguishing characteristics that are indicative of an involuntary, subconscious response of the brain of the person to a stimulus belonging to one of the stimuli categories of interest. Stimuli in each of the categories produce different distinguishing characteristics. The input signals are employed to train the detection module to recognize the aforementioned distinguishing characteristics exhibited within the signals for each of the stimuli categories of interest. Once the detection module is trained, a stimulus is presented to the person whose brain activity is being monitored, and signals from the brain activity sensing device are input. The detection module is used to recognize the aforementioned distinguishing characteristics whenever they are exhibited in the signals. The detection module then outputs an indicator identifying the stimuli category to which the currently presented stimulus belongs.

In some embodiments of the present technique, a stimulus is presented multiple times to the same person, or to multiple people, in the training or detection phases, in order to make the technique more robust.

In addition to the just described benefits, other advantages of the present invention will become apparent from the detailed description which follows hereinafter when taken in conjunction with the drawing figures which accompany it.

### DESCRIPTION OF THE DRAWINGS

The specific features, aspects, and advantages of the present invention will become better understood with regard to the following description, appended claims, and accompanying drawings where:

FIG. 1 is a diagram depicting a general purpose computing device constituting an exemplary system for implementing the present invention.

FIG. 2 is a flow diagram generally outlining an embodiment of a process for identifying the stimuli category of a stimulus that has been presented to a person being monitored with a brain activity sensing device.

FIG. 3 is a diagram depicting the layout of an electroencephalograph (EEG) device's electrodes on a person's scalp as defined by the International 10-20 electrode placement standard.

FIG. 4 is a graph plotting the average Event-Related Potential (ERP) response signals output by an EEG device versus time after the presentation of a non-face image to a person being monitored.

FIG. 5 is a graph plotting the average ERP response signals output by an EEG device versus time after the presentation of a face image to a person being monitored.

FIG. 6 is a flow diagram generally outlining an embodiment of a process for training a detection module to recognize the distinguishing signal characteristics associated with a stimulus belonging to each of one or more stimuli categories of interest in accordance with the present perceptual stimulus categorization technique.

FIG. 7 is a flow diagram generally outlining an embodiment of a process for using the trained detection module to detect if a stimulus from a trained stimuli category is presented to the person being monitored and to identify the category in accordance with the present perceptual stimulus categorization technique.

FIG. 8 is a flow diagram generally outlining an embodiment of a process for determining when the brain activity sensing device signals are considered to be exhibiting the distinguishing characteristics associated with a stimuli category in accordance with the present perceptual stimulus categorization technique using a voting scheme approach.

FIG. 9 is a flow diagram generally outlining an embodiment of a process for determining when the brain activity sensing device signals are considered to be exhibiting the distinguishing characteristics associated with a stimuli category in accordance with the present perceptual stimulus categorization technique using a weighted indicator approach.

FIG. 10 is a flow diagram generally outlining an embodiment of a process for detecting whether a stimulus that is presented to a person being monitored multiple times belongs to a trained stimuli category and outputting an indicator identifying the category in accordance with the present perceptual stimulus categorization technique.

FIG. 11 is a flow diagram generally outlining an embodiment of a process for using a voting scheme approach to determine which trained stimuli category a stimulus that is presented to a person multiple times belongs to in accordance with the process of FIG. 10.

FIG. 12 is a flow diagram generally outlining an embodiment of a process for using a weighted indicator approach to determine which trained stimuli category a stimulus that is presented to a person multiple times belongs to in accordance with the process of FIG. 10.

### DETAILED DESCRIPTION

In the following description of embodiments of the present invention reference is made to the accompanying drawings which form a part hereof, and in which are shown, by way of illustration, specific embodiments in which the invention may be practiced. It is understood that other embodiments may be utilized and structural changes may be made without departing from the scope of the present invention.

### 1.0 The Computing Environment

Before providing a description of embodiments of the present perceptual stimulus categorization technique, a brief, general description of a suitable computing environment in which portions thereof may be implemented will be described. The present technique is operational with numerous general purpose or special purpose computing system environments or configurations. Examples of well known computing systems, environments, and/or configurations that may be suitable include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like.

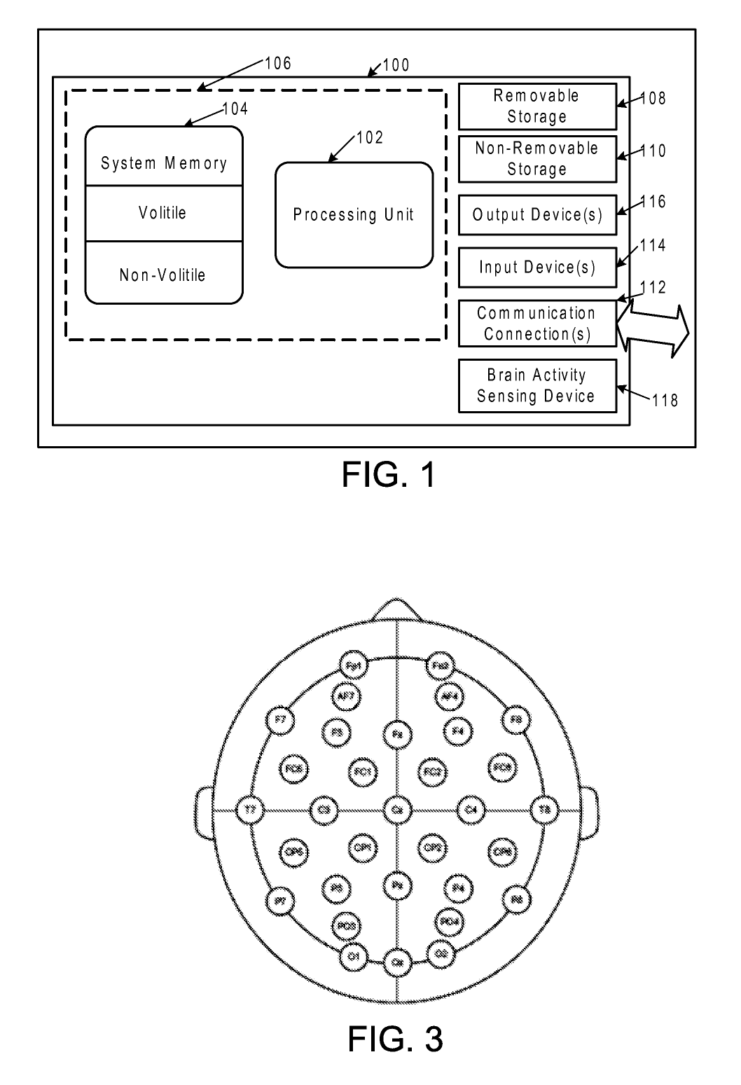

FIG. 1 illustrates an example of a suitable computing system environment. The computing system environment is only one example of a suitable computing environment and is not intended to suggest any limitation as to the scope of use or functionality of the present perceptual stimulus categorization technique. Neither should the computing environment be interpreted as having any dependency or requirement relating to any one or combination of components illustrated in the exemplary operating environment. With reference to FIG. 1, an exemplary system for implementing the present technique includes a computing device, such as computing device 100. In its most basic configuration, computing device 100 typically includes at least one processing unit 102 and memory 104. Depending on the exact configuration and type of computing device, memory 104 may be volatile (such as RAM), non-volatile (such as ROM, flash memory, etc.) or some combination of the two. This most basic configuration is illustrated in FIG. 1 by dashed line 106. Additionally, device 100 may also have additional features/functionality. For example, device 100 may also include additional storage (removable and/or non-removable) including, but not limited to, magnetic or optical disks or tape. Such additional storage is illustrated in FIG. 1 by removable storage 108 and non-removable storage 110. Computer storage media includes volatile and nonvolatile, removable and non-removable media implemented in any method or technology for storage of information such as computer readable instructions, data structures, program modules or other data. Memory 104, removable storage 108 and non-removable storage 110 are all examples of computer storage media. Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can accessed by device 100. Any such computer storage media may be part of device 100.

Device 100 may also contain communications connection(s) 112 that allow the device to communicate with other devices. Communications connection(s) 112 is an example of communication media. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. The term computer readable media as used herein includes both storage media and communication media.

Device 100 may also have input device(s) 114 such as keyboard, mouse, pen, voice input device, touch input device, camera, etc. Output device(s) 116 such as a display, speakers, printer, etc. may also be included. All these devices are well know in the art and need not be discussed at length here.

Of particular note is that device 100 can include a brain activity sensing device 118, which is capable of measuring brain activity, as an input device. The activity information from the sensing device 118 is input into the device 100 via an appropriate interface (not shown). However, it is noted that brain activity data can also be input into the device 100 from any computer-readable media as well, without requiring the use of the brain activity sensing device.

The present perceptual stimulus categorization technique may be described in the general context of computer-executable instructions, such as program modules, being executed by a computing device. Generally, program modules include routines, programs, objects, components, data structures, etc. that perform particular tasks or implement particular abstract data types. The present technique may also be practiced in distributed computing environments where tasks are performed by remote processing devices that are linked through a communications network. In a distributed computing environment, program modules may be located in both local and remote computer storage media including memory storage devices.

The exemplary operating environment having now been discussed, the remaining parts of this description section will be devoted to a description of the program modules embodying the present perceptual stimulus categorization technique.

### 2.0 The Perceptual Stimulus Categorization Technique

The present perceptual stimulus categorization technique uses brain sensing technology to detect involuntary, subconscious processing performed by the brain in order to perform useful tasks. For instance, when a visual, auditory or some other type of stimulus is presented to a person whose brain activity is being monitored, the person's subconscious response can be detected and used for recognition purposes.

One example of this is object or face recognition using an image as the stimulus. It has been found that presenting images of different classes of stimuli (e.g., faces, cars, animals, mushrooms, chairs, and so on) evoke different subconscious responses. In the case of human faces, for example, functional Magnetic Resonance Imaging (fMRI) studies show characteristic activation of certain region of the brain, popularly known as the fusiform face area. Furthermore, Electroencephalograph (EEG) studies show similarly characteristic properties in the signals produced when the brain processes faces. The characteristic activation can be sensed and used as an indication that a subject has been shown an image of a face, as will be described in more detail later.

As alluded to previously, the person whose brain activity is being monitored does not need to be consciously aware that a stimulus has been presented. The subconscious response occurs anyway. Thus, the stimulus could be placed in the person's attentive (e.g., visual or audio) periphery. This would allow a person to go about other tasks as they normally would without interruption. It is also envisioned that a stimulus could be presented multiple times to the same person or to multiple people to redundantly process information, as will be described in more detail later. This redundancy would make the process more robust.

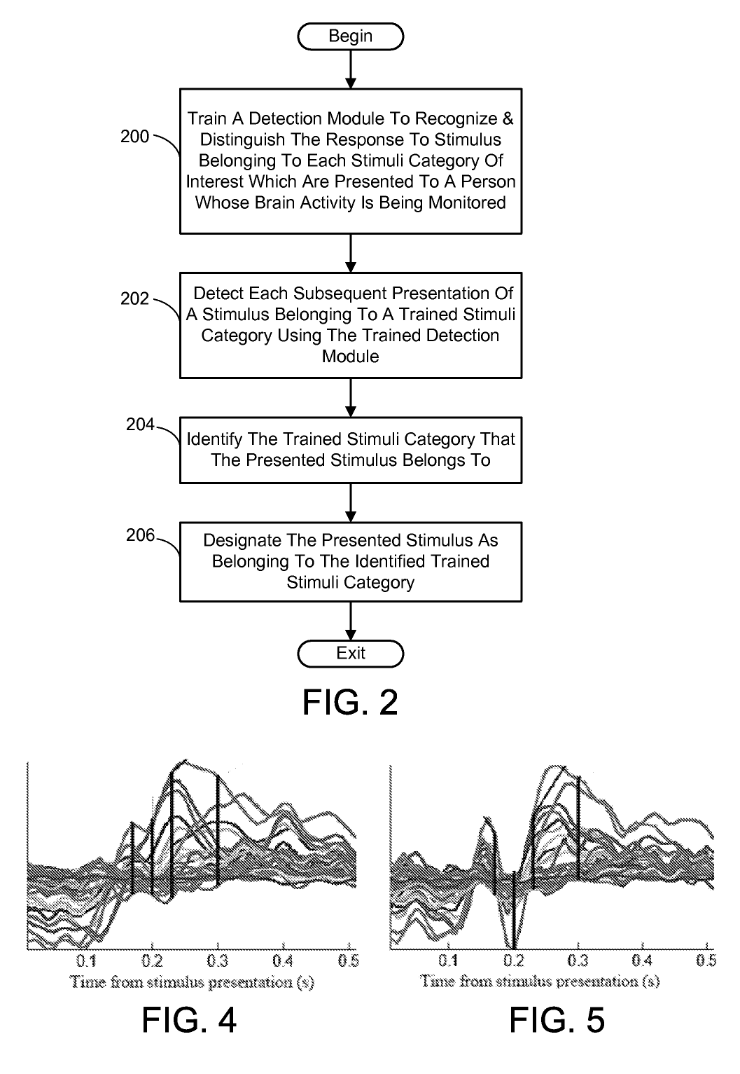

In general, the present perceptual stimulus categorization technique involves detecting whether a stimulus belonging to one or more categories of stimuli has been presented to a person whose brain activity is being monitored and then identifying the category. Referring to FIG. 2, the technique generally includes three phases. First, a training phase 200 involves obtaining signals indicative of brain activity while stimuli known to belong to the one or more categories of interest are presented to the person being monitored, and training a detection module to recognize and distinguish the part of the brain activity signals generated in response to a stimulus belonging to each stimuli category of interest. Then, a detection phase 202 involves, once the detection module is trained, detecting a subsequent instance or instances of a stimulus belonging to a trained stimuli category being presented to the monitored individual. This is again accomplished using the obtained brain activity signals. The trained stimuli category that the presented stimulus belongs to is then identified (204). Finally, a designation phase 206 involves designating the presented stimulus as belonging to its identified trained stimuli category.

Each of these general phases of the present technique will be described in more detail in the following sections, after first describing how the brain activity signals can be obtained.

### 2.1 Obtaining Brain Activity Signals

Various brain activity sensing devices are available for obtaining signals indicative of brain activity, including devices that require invasive procedures such as electrocorticography (ECoG), use large equipment such as functional magnetic resonance imaging (fMRI) and magnetoencephalography (MEG), or wearable devices such as functional near infrared spectrographs (fNIRs) and electroencephalograph (EEG) devices, among others. Generally, these devices produce output signals representing the state of brain activity in different parts of the brain. While any type of brain activity sensing device can be employed in the present technique, the following description will use an EEG device as an example.

An EEG device uses electrodes placed on the scalp to measure electrical potentials related to brain activity. Each electrode consists of a wire leading to a conductive disk that is electrically connected to the scalp using conductive paste or gel. Traditionally, the EEG device records the voltage at each of these electrodes relative to a reference point (often another electrode on the scalp). Electrode placements on the scalp are typically defined by the International 10-20 electrode placement standard. Because EEG is a non-invasive, passive measuring device, it is safe for extended and repeated use.

The signal provided by an EEG is, at best, a crude representation of brain activity due to the nature of the detector. Scalp electrodes are only sensitive to macroscopic coordinated firing of large groups of neurons near the surface of the brain, and then only when they are directed along a vector perpendicular to the scalp. Additionally, because of the fluid, bone, and skin that separate the electrodes from the actual electrical activity, the already small signals are scattered and attenuated before reaching the electrodes. Despite this, EEG data is still a useful way to monitor changes in brain activity, such as occur when a person is presented with an environmental stimulus.

One way to analyze EEG data is to look at the spectral power of the signal in a set of frequency bands, which have been observed to correspond with certain types of neural activity. These frequency bands are commonly defined as 1-4 Hz (delta), 4-8 Hz (theta), 8-12 Hz (alpha), 12-20 Hz (beta-low), 20-30 Hz (beta-high), and >30 Hz (gamma). Another way the EEG signal can be analyzed is by inspecting the Event-Related Potential (ERP). This is the spatiotemporal pattern of EEG signals produced in response to discrete visual, auditory or other stimuli. The idea is that different kinds of discrete stimuli evoke characteristic, different ERPs, which can be detected in the shape of the raw data.

In the context of the present technique, an EEG can be employed as the brain activity sensing device and the ERP signals can be used to detect whether a stimulus from a particular category of stimuli has been presented to a person being monitored. It is noted that in tested embodiments, the ERP signals represent the potential difference between each electrode pair, rather than in reference to a single reference electrode as is typically the case.

The number of electrode pairs employed and the electrode placement can vary as desired. However, in tested embodiments of the present technique, the aforementioned International 10-20 electrode placement standard was used (as illustrated in FIG. 3) and the following exemplary electrode set was monitored: T7, T8, P3, PZ, P4, P7, P8, PO3, PO4, O1, Oz and O2.

The EEG signals were also pre-processed in the tested embodiments to streamline the procedure. First, the raw voltage signal read from each electrode pair was converted to a digital signal. Each of the resulting digital signals was then sampled to reduce the amount of processing necessary to analyze the signals. In the tested embodiments, this entailed downsampling each signal to 100 Hz. This 100 Hz signal is more than sufficient for the purposes of the present technique. The sampled signals are then converted into the frequency domain using any appropriate transformation technique (e.g., FFT, MCLT, and so on). It is believed that the subconscious processing of environmental stimuli is exhibited in EEG signals in a particular frequency bandwidth between about 0.15 and about 30 Hz. Accordingly, to simplify the analysis of the EEG signals, bandpass filtering was employed in the tested embodiments to retain only the frequency band of interest. While any bandpass filtering technique can be employed, Finite Impulse Response (FIR) filtering was employed in the tested embodiments as this type of filtering is inherently stable and computationally efficient.

It is noted that the changes in brain activity that can be attributed to the presentation of a particular category of stimuli will often occur in a specific time period after the presentation. If this time period is known and it is also known when a stimulus is presented to the person being monitored (as will often be the case), then the pre-processing procedure can include a step of retaining only the portion of the signals occurring within the appropriate period of time after the stimulus is presented to the person. For example, FIGS. 4 and 5, respectively shows graphs of monitored EEG signals for a period of 0.5 seconds after an image of a person's face was presented to a subject (FIG. 5) and after a non-face image was presented (FIG. 4). Notice the readily discernable dip in some of the signals associated with the face image stimulus at about 170 milliseconds. Thus, if the task is to have a subject subconsciously identify face images, only the part of the signal surrounding the 170 millisecond time would be needed for the training and detection phases. Thus, for example, the EEG signal could be cropped to a 100-300 millisecond time window following stimulus presentation. On the other hand, if it is not known when a discernable change in the EEG signals will occur in response to a stimulus of interest, or if the identifying pattern is spread out across the subconscious processing period, then the EEG signals would not be cropped.

### 2.2 Training The Detection Module

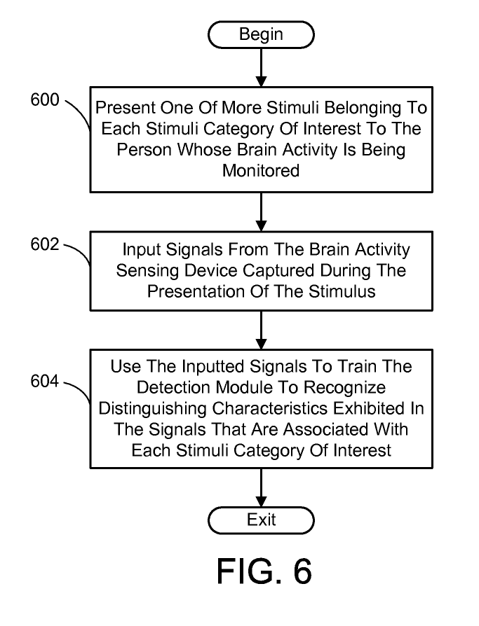

The training phase is accomplished prior to presenting stimuli to a person for detection purposes. In general, referring to FIG. 6, the training of the detection module entails first presenting to the person being monitored, one or more stimuli belonging to each of the stimuli categories of interest that will later be presented to the person during the detection phase (600). Signals from the brain activity sensing device being employed are input during the presentation of the aforementioned stimuli (602). As described previously, it is assumed the signals will exhibit distinguishing characteristics when a stimulus belonging to the one or more stimuli categories of interest is presented to the person being monitored. These distinguishing characteristics are a consequence of the involuntary, subconscious response of the brain of the person to the stimulus and will be different for each different stimuli category. The inputted signals are then used to train the detection module to recognize the respective distinguishing characteristics exhibited in the signals for each of the stimuli categories of interest (604).

Analysis techniques that focus on general distinguishing characteristics (or data features) of the EEG signal extract the data contained in the EEG signals and pass the data to machine learning algorithms. Focusing on general data features of the EEG signal allows the machine learning algorithms to treat the brain as a black box in that no information about the brain or the user goes into the analysis. The analysis usually involves signal processing and may also involve data feature generation, i.e., feature generation, and selection of relevant data features, i.e., features.

Machine learning classifier techniques are used to classify the results. A machine learning classifier is a function that maps an input attribute vector, x=(x1, x2, x3, x4, . . . , xn), to a confidence that the input belongs to a class, that is, f(x)=confidence(class). Such classification can employ a probabilistic and/or statistical-based analysis (e.g., factoring into the analysis utilities and costs) to prognose or infer an action that a user desires to be automatically performed. A support vector machine (SVM) is an example of a classifier that can be employed. The SVM operates by finding a hypersurface in the space of possible inputs, which the hypersurface attempts to split the triggering criteria from the non-triggering events. Intuitively, this makes the classification correct for testing data that is near, but not identical to training data. Other directed and undirected model classification approaches include, e.g., naïve Bayes, Bayesian networks, decision trees, neural networks, decision trees, genetic algorithms, fuzzy logic models, and probabilistic classification models providing different patterns of independence can be employed. Classification as used herein also is inclusive of statistical regression that is utilized to develop models of priority.

Machine learning techniques acquire knowledge automatically from examples, i.e., from source data as opposed to performance systems, which acquire knowledge from human experts. Machine learning techniques enable systems, e.g., computing devices, to autonomously acquire and integrate knowledge, i.e., learn from experience, analytical observation, etc., resulting in systems that can continuously self-improve and thereby offer increased efficiency and effectiveness.

In general, these training techniques involve capturing the signals output from the brain activity sensing device for a prescribed period of time when a stimulus known to belong to each of the one or more stimuli categories of interest is been presented to the person. The captured signals associated with each stimuli category of interest are then used to train the detection module to recognize distinguishing characteristics that are uniquely indicative of a stimulus belonging to one of the aforementioned categories.

In the case of the tested embodiments employing an EEG as the brain activity sensing device, a spatial projection algorithm was employed in the training process. This algorithm projects the response sequences from the multiple signals onto three maximally discriminative time series. A linear discriminant analysis (LDA), which is a type of supervised machine learning method, is then used to classify the resulting features into mutually exclusive and exhaustive groups.

It is noted that the stimulus presented to a person being monitored can be presented in a way that it comes to the cognitive attention of the individual, although that individual would not know why it is being presented. For example, the subject could be told to watch a series of images, but not told what particular type of image they are looking for. Since subconscious responses are what is being trained (and later detected) it is not required that the person know what type of images they are looking for, even though they are aware of each image shown. Further, the subject need not become aware of the stimulus. Thus, it could be presented in such a way as it could stay in the attentive periphery of the subject. This is possible as the stimulus will still produce the same subconscious response, regardless of whether the person becomes aware of it or not.

### 2.3 Detecting The Presentation Of A Stimulus And Designating The Trained Stimuli Category It Belongs To

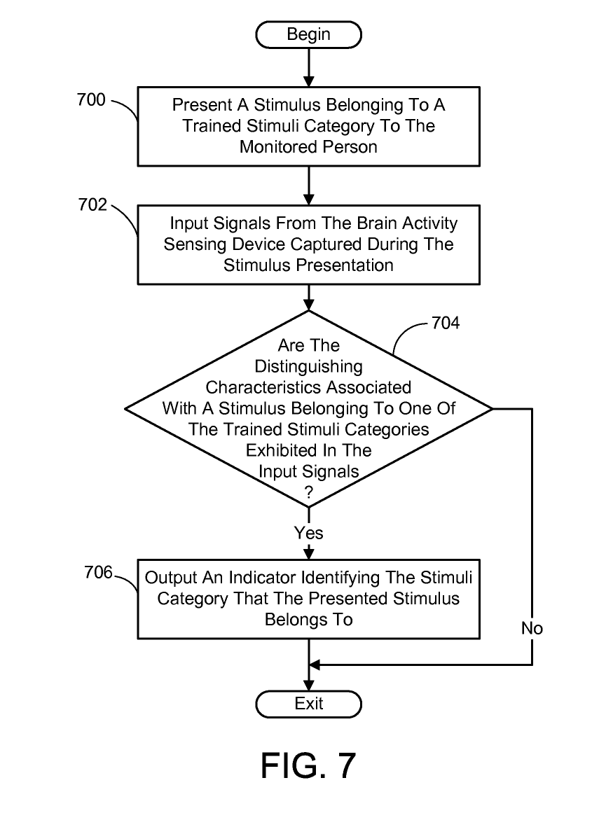

Once the detection module is trained to recognize the distinguishing signal characteristics associated with a stimulus belonging to each of the stimuli categories of interest, it can be used to detect if a stimulus from one of these categories is presented to the person being monitored. To this end, referring to FIG. 7, in one embodiment, the detection phase involves first presenting a stimulus belonging to a trained stimuli category to the person (700) while the individual is being monitored by the brain activity sensing device. The signals from the device are input (702) and the detection module is used to determine if distinguishing characteristics associated with a stimulus belonging to one of the trained stimuli categories are present in the brain activity sensing device signals (704). Conventional methods are employed to analyze the signals and recognize the aforementioned distinguishing characteristics whenever they are exhibited in the signals with a technique appropriate to the particular machine learning algorithm that has been used. If it is determined the distinguishing characteristics associated with one of the trained stimuli categories are present in the signals, then an indicator is output identifying the stimuli category that the presented stimulus belongs to (706).

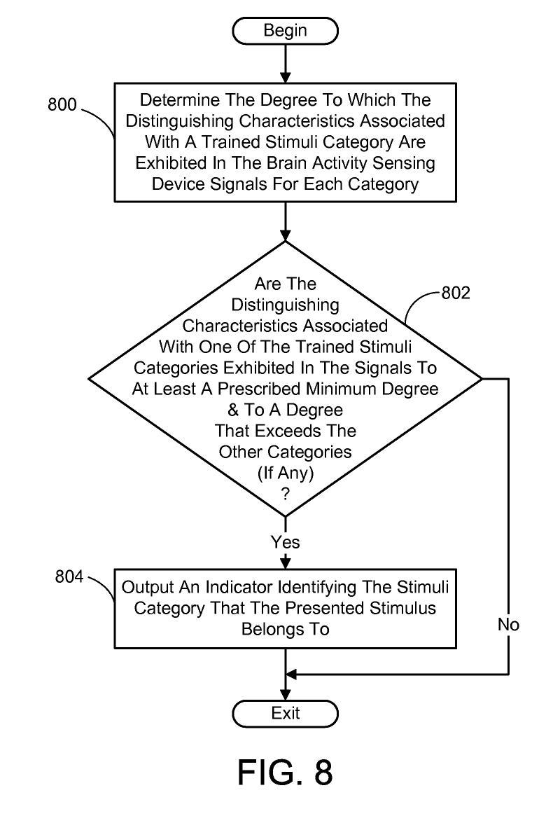

In regard to when the brain activity sensing device signals are considered to be exhibiting the distinguishing characteristics associated with a trained stimuli category, this can be done in different ways. Referring to FIG. 8, in one embodiment the degree to which the aforementioned distinguishing characteristics associated with a trained stimuli category are exhibited in the brain activity sensing device signals is determined for each stimuli category (800). A stimulus presented to the monitored person is deemed to belong to a trained stimuli category whenever the distinguishing characteristics are determined to be exhibited to some prescribed minimum degree (e.g., 60% in a 2-class scenario or 40% in a 3-class scenario) which exceeds the other categories (if any). Thus, it is next determined if distinguishing characteristics associated with one of the trained stimuli categories are exhibited in the signals to at least the prescribed minimum degree and the degree to which they are exhibited exceeds the others categories (if any) (802). If so, an indicator is output identifying the stimuli category that the presented stimulus belongs to (804). If not, either no indicator or a default indicator is output.

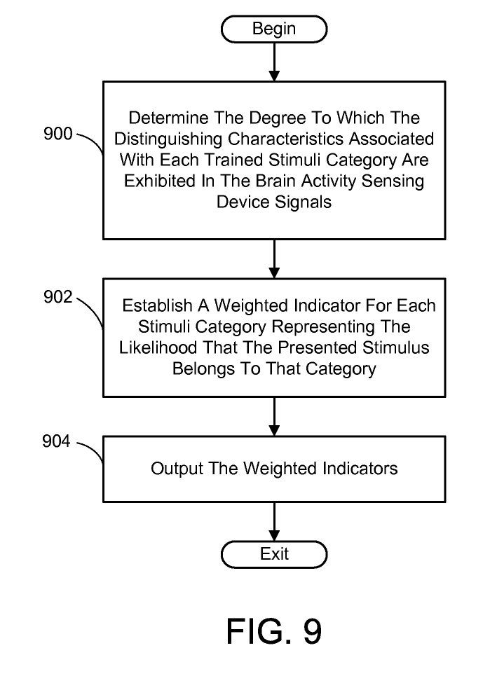

Referring to FIG. 9, in another version of the present technique, whenever a stimulus is presented to a monitored person, the degree to which the aforementioned distinguishing characteristics associated with each trained stimuli category are exhibited in the brain activity sensing device signals is determined for each stimuli category (900) as before. However, in this version, a weighted indicator would be established for each stimuli category, whose weight represents the likelihood that the stimulus presented to the person belongs to a trained stimuli category (902). For example, a weighted indicator might indicate that the likelihood that the distinguishing characteristics are exhibited in the signals is 60 percent in one category and 15 percent in another. In this version, when it is determined the distinguishing characteristics are not exhibited to some prescribed minimum degree, the weighted indicator can simply be a zero. The weighted indicator(s) are then output (904). It is noted that in this version, the designation as to which of the trained categories the stimulus belongs to is done after the detection module outputs the weighted indicators, rather than being done by the module itself. The determination procedure can be similar though in that the trained stimuli category associated with the largest weighted indicator is designated as the category of the presented stimulus. However, as will be described in the next section, the determination can involve more.

### 2.4 Increasing The Robustness Of The Technique

As mentioned previously, a stimulus could be presented multiple times to the same person, or to multiple people, in order to make the present perceptual stimulus categorization technique more robust. In regard to the training phase, some of the aforementioned training methods would accommodate presenting a stimulus multiple times during the training of the detection module. The resulting signals produced at each presentation would be used to create potentially more reliable categorization of the stimuli associated with the categories of interest. Thus, in this multiple stimulus training embodiment, each stimulus associated with each of the categories of interest would be presented to the monitored person multiple times during the training phase. In regard to the detection phase, the multiple stimulus presentation and multiple person presentation features will now be described in more detail in the sections to follow.

### 2.4.1 Presenting A Stimulus Multiple Times

In one embodiment of the present technique, the multiple stimulus presentation feature is implemented as follows. The training phase is the same as described in connection with FIG. 6, or can involve the multiple stimulus presentation scheme mentioned above. However, the detection and designation phases diverge from the previously described embodiments because each stimulus is presented multiple times to the person being monitored.

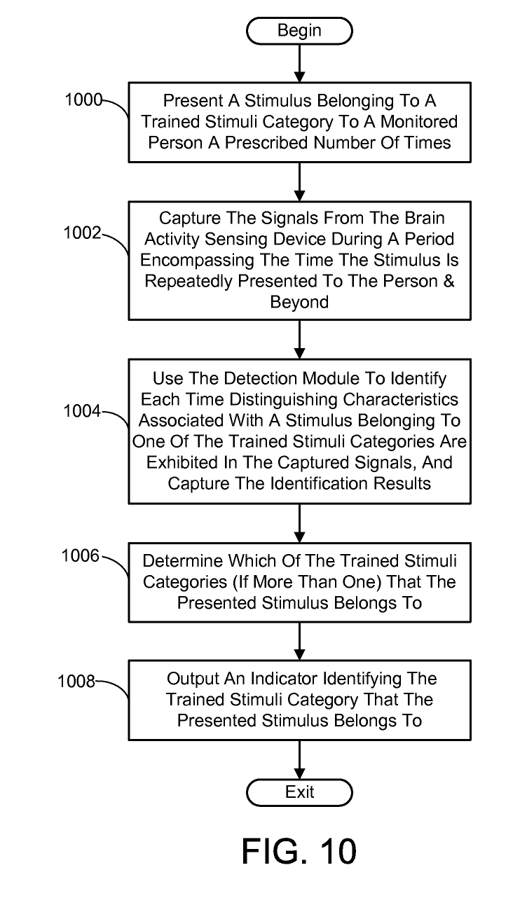

More particularly, as shown in FIG. 10, in the detection phase, a stimulus belonging to a trained stimuli category is presented to the person repeatedly for a prescribed number of times (1000). For example, in tested embodiments, a stimulus was presented between one and ten times to a given user. The signals from the brain activity sensing device are captured during a period encompassing the time the stimulus is repeatedly presented to the person and beyond (1002). The detection module is used to identify each time distinguishing characteristics associated with a stimulus belonging to one of the trained stimuli categories are present in the captured signals and capturing the results of the identifications (1004). It is then determined which of the trained stimuli categories (if there are more than one) that the presented stimulus belongs to based on the identification results (1006). Then, in the designation phase, an indicator identifying the trained stimuli category that the presented stimulus belongs to is output (1008).

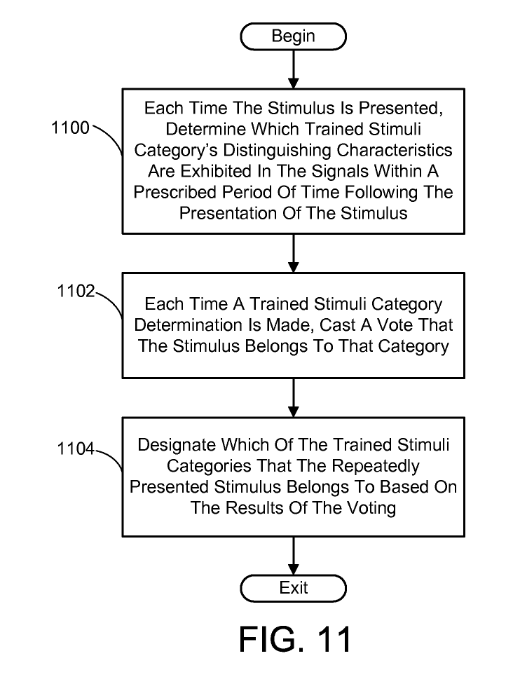

In one embodiment of the present technique, determining if a stimulus that is repeatedly presented to a person being monitored belongs to one of the trained stimuli categories, is accomplished as follows. Referring to FIG. 11, for each time the stimulus is presented to the person being monitored, it is determined which trained stimuli category's distinguishing characteristics are exhibited in the brain activity sensing device signals within a prescribed period of time following the presentation of the stimulus (1100). A voting scheme can then be employed. More particularly, for each instance where distinguishing characteristics associated with a trained stimuli category are exhibited in the signals, a vote is cast that the stimulus belongs to that category (1102). Based on the results of the voting, it is then designated which of the trained stimuli categories the repeatedly presented stimulus belongs to (1104). In one embodiment, the top vote-getter wins.

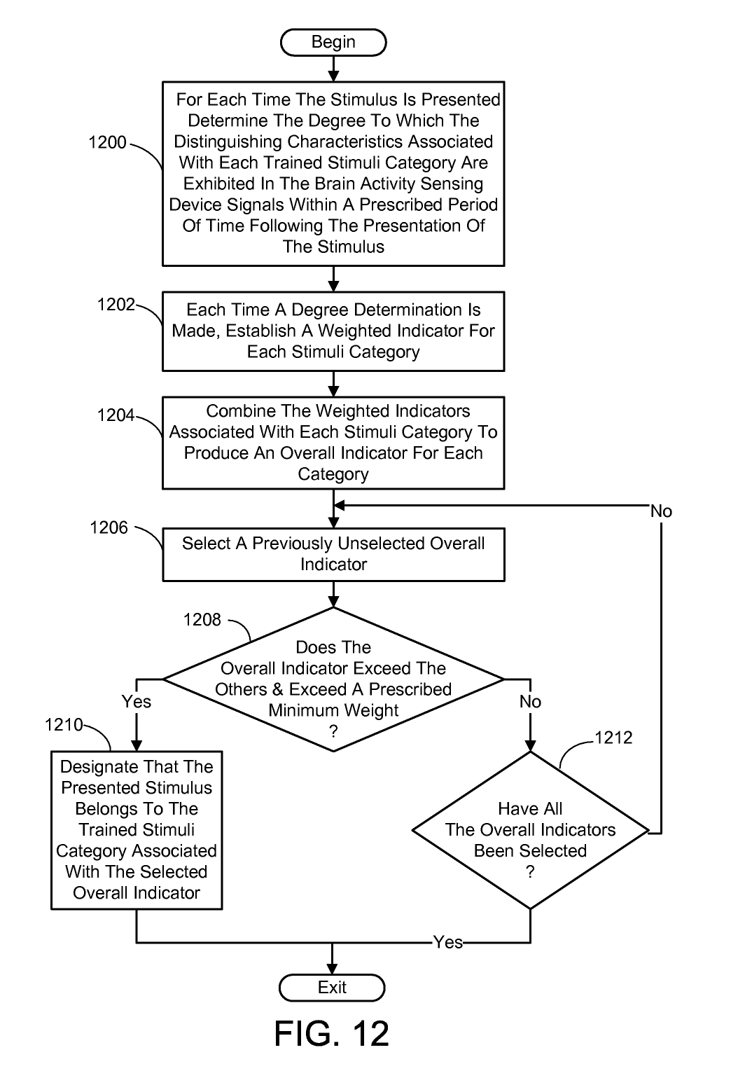

In another embodiment of the present technique, determining if a stimulus repeatedly presented to the person being monitored belongs to one of the trained stimuli categories is accomplished as follows. Referring to FIG. 12, for each time the stimulus is presented to the person being monitored, the degree to which the aforementioned distinguishing characteristics associated with each trained stimuli category are exhibited in the brain activity sensing device signals within a prescribed period of time following the presentation of the stimulus, is determined (1200). Next, a weighted indicator is established for each stimuli category and each instance of the stimulus being presented to the person being monitored (1202). The weight of this weighted indicator represents the likelihood that the stimulus presented to the person belongs to a trained category of stimuli. In this embodiment, when it is determined the distinguishing characteristics are not exhibited to at least a minimum degree, the weighted indicator can simply be a zero for that category. The weighted indicators associated with all the presentation instances for each trained stimuli category are then combined (e.g. simply by averaging them together, or in a more complex manner by decaying the importance of indicators based on time or other factors before taking the normalized average) to produce an overall indicator for each category (1204). A previously unselected overall indicator is then selected (1206). It is next determined if the selected overall indicator exceeds the others (if there are more than one), and exceeds a prescribed minimum weight (e.g., 60% in a 2-class scenario or 40% in a 3-class scenario) (1208). If so, it is then designated that the stimulus presented to the person was a stimulus belonging to the trained stimuli category associated with the identified (“winning”) overall indicator (1210), and the process ends. If not, it is determined if all the overall indicators have been selected (1212). If there are some remaining, then actions 1206 through 1212 are repeated. Otherwise, the process ends.

In yet another embodiment of the present technique, the system could dynamically determine if and when a particular stimulus should be presented again before it is determined which category it belongs to. In the simplest scenario, the user may have a certain amount of time for a certain number of stimuli to be presented (e.g. 50) and the system may need some other number of unique stimuli to be categorized (e.g. 40). In this case, the system has to decide which of the 40 images to show again. In the simplest instantiation, the system could re-show the stimuli that have received the lowest weighted winning indictors, or it could re-show the ones that have the smallest difference between the highest (winning) and second highest indicators, since these categorizations are the most likely to change with a second presentation. In another scenario, the system could continue to re-show stimuli until some confidence threshold for that indicator is reached (i.e. the weighted indicator for a category goes above a certain limit or the difference between the highest (winning) and second highest indicators are above some threshold).

### 2.4.2 Presenting A Stimulus To Multiple People

In one embodiment of the present technique, the multiple person presentation feature is implemented as follows. Generally, the training phase (which can involve either the previously-described single or multiple presentation scenarios) and the detection phase are the same as described previously for a single person (see FIGS. 2, 6 and 7), except they are repeated for each additional person being monitored. As a result, detection data is generated for each person. It is the designation phase that is significantly different when the multiple person presentation feature is implemented.

More particularly, in one embodiment where the detection data is in the form of a detection indicator identifying the stimuli category that the stimulus belongs to, a voting scheme is employed to make the ultimate determination of the presented stimulus's category. The voting scheme involves casting a vote that the stimulus presented to the people being monitored belongs to a particular trained stimuli category for each indicator output which identifies that category. Based on the results of the voting, it is then designated which stimuli category the stimulus presented to the people being monitored belongs to. In one embodiment, the highest vote-getter wins.

In another embodiment of the present technique, the detection data from each person is in the form of a weighted indicator for each of the trained categories, whose weights represent the likelihood that the stimulus presented to a person belongs to a category. In this embodiment, when it is determined the distinguishing characteristics are not exhibited, the weighted indicator is a zero. The multiple person presentation feature can be implemented in this weighted indicator embodiment as follows. First, the weighted indicators associated with each of the trained stimuli categories are respectively combined as in the single-user multiple-presentation case discussed above to produce an overall indicator for each category. It is next identified which of the overall indicators exceeds the others and exceeds a prescribed minimum as before. It is then designated that the stimulus presented to the people was a stimulus belonging to the trained stimuli category associated with the identified (“winning”) overall indicator.

### 3.0 Other Embodiments

It is noted that although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

It should also be noted that any or all of the aforementioned embodiments throughout the description may be used in any combination desired to form additional hybrid embodiments. For example, the multiple stimulus presentation and multiple person presentation features associated with the detection and designation phases could be combined such that each of the multiple people presented with a stimulus is presented with the stimulus multiple times. In this hybrid embodiment, the results of presenting the stimulus multiple times to each person involved would then be combined to produce a final indication of which trained stimuli category the presented stimulus belongs to.

In the case where the results of presenting the stimulus to a person multiple times is a single indicator identifying the stimuli category the stimulus belongs to, the results would be combined using a voting scheme. This scheme involves casting a vote that the stimulus presented to the people belongs to a particular trained stimuli category for each indicator which identifies that category. Based on the results of the voting, it is then designated which stimuli category the stimulus presented to the people belongs to. For example, the highest vote-getter could win.

In the case where the results of presenting the stimulus to a person multiple times is a weighted indicator for each of the trained stimuli categories, in one embodiment the results would be combined in the following manner. The weighted indicators associated with each trained stimuli category would be respectively combined to produce an overall indicator for each category. It is next identified which of the overall indicators exceeds the others and exceeds a prescribed minimum as before. It is then designated that the stimulus presented to the people was a stimulus belonging to the trained stimuli category associated with the identified (“winning”) overall indicator.

In another embodiment involving the weighted indicators, the results of presenting the stimulus to a person multiple times would be combined for all the people involved in the following manner. The weighted indicators associated with all the presentation instances for each trained stimuli category for a monitored person would be combined to produce an overall indicator for each category. It would then be determined if one of the overall indicators exceeds the others and exceeds a prescribed minimum value as before. If so, it is designated that the stimulus presented to the person was a stimulus belonging to the trained stimuli category associated with the identified (“winning”) overall indicator. This is repeated for all the people. A voting scheme is then employed where a vote is cast that the stimulus presented to the people belongs to a particular trained stimuli category for each indicator which identifies that category. Based on the results of the voting, it is then designated which stimuli category the stimulus presented to the people being monitored belongs to. For example, the highest vote-getter could win.

Wherefore, what is claimed is:

1. A computer-implemented process for identifying a stimuli category of a perceptual stimulus that has been presented to one or more people whose brain activity is being monitored, comprising using a computer to perform the following process actions for each person:
training a detection module to recognize the part of the brain activity generated in response to a presentation of a stimulus belonging to each of one or more categories of perceptual stimuli using the monitored brain activity;
once the detection module is trained, detecting a subsequent instance or instances of a stimulus belonging to a trained stimuli category being presented to the person based on the monitored brain activity;
identifying the trained stimuli category that the presented stimulus belongs to; and
designating that the presented stimulus belongs to the identified trained stimuli category.

2. A computer-implemented process for identifying a stimuli category of a perceptual stimulus that has been presented to one or more people whose brain activity is being monitored, comprising using a computer to perform the following process actions for each person:
prior to presenting the stimulus to the person,
presenting to the person, at least once, a training stimulus belonging to each of one or more stimuli categories of interest,
inputting signals from a brain activity sensing device captured during a time the training stimulus was presented and beyond, wherein the signals exhibit different distinguishing characteristics whenever a stimulus belonging to a different one of the one or more stimuli categories is presented to the person, and wherein the distinguishing characteristics are indicative of an involuntary, subconscious response of the brain of the person to the stimulus, and
employing the inputted signals to train a detection module to recognize the respective distinguishing characteristics associated with each of the stimuli categories of interest; and
once the detection module is trained,
presenting a stimulus belonging to a trained stimuli category to the person,
inputting signals from the brain activity sensing device captured during a time the stimulus was presented and beyond,
using the detection module to determine if distinguishing characteristics associated with a stimulus belonging to one of the trained stimuli categories are present in the inputted signal, and
outputting an indicator identifying the trained stimuli category that the stimulus belongs to, whenever it is determined distinguishing characteristics associated with a stimulus belonging to one of the trained stimuli categories are present in the inputted signal.

3. The process of claim 2, wherein it is known that the involuntary, subconscious response of the brain of the person to a stimulus belonging to a stimuli category will occur within a particular period of time after the stimulus is presented to the person, and wherein signals from the brain activity sensing device are captured for this particular period of time after the stimulus has been presented.

4. The process of claim 2, wherein the brain activity sensing device is an electroencephalograph (EEG) which produces multiple signals representing the difference in potential over time between pairs of sensors each of which is placed at a different location on the scalp of the person, and wherein the process actions of inputting signals from the brain activity sensing device comprises the actions of:
inputting the multiple signals from the EEG; and
pre-processing the signals prior to providing them to the detection module.

5. The process of claim 4, wherein the process action of pre-processing the signals, comprises the actions of:
converting each of the EEG signals associated with pairs of sensors of interest to a digital signal;
sampling each of the resulting digital signals at a prescribed rate to reduce the amount of processing necessary to analyze the signals;
transforming each sampled signal to the frequency domain using a prescribed transformation technique; and
bandpass filtering each of the transformed signals to eliminate frequencies above a prescribed upper limit and below a prescribed lower limit.

6. The process of claim 4, wherein it is known that the involuntary, subconscious response of the brain of the person to a stimulus belonging to a stimuli category will occur within a particular period of time after the stimulus is presented to the person, and wherein the process action of pre-processing the signals, further comprises an action of retaining only the portion of the signals occurring within the particular period of time after the stimulus is presented to the person.

7. The process of claim 2, wherein each time a stimulus is presented to the person, it is presented in such a way as the person is attentive to the stimulus.

8. The process of claim 2, wherein each time a stimulus is presented to the person, it is presented in such a way that it is in the person's attentive periphery and so the person is not conscious of the stimulus.

9. The process of claim 2, wherein the process action of outputting an indicator identifying the trained stimuli category that the stimulus belongs to, comprises the actions of:
for each of the one or more stimuli categories, determining the degree to which distinguishing characteristics associated with the stimuli category under consideration are exhibited in the signals;
determining if the distinguishing characteristics associated with one of the trained stimuli categories are exhibited in the signals to at least a prescribed degree and to a degree that exceeds the other categories, if any; and
outputting the indicator identifying the stimuli category that the stimulus presented to the person belongs to, whenever it is determined the distinguishing characteristics associated with one of the trained stimuli categories are exhibited in the signals to at least the prescribed degree and to a degree that exceeds the other categories, if any.

10. The process of claim 2, wherein the process action of outputting an indicator identifying the trained stimuli category that the stimulus belongs to, comprises the actions of:
for each of the one or more stimuli categories, determining the degree to which distinguishing characteristics associated with the stimuli category under consideration are exhibited in the signals;
whenever distinguishing characteristics associated with a trained stimuli category are exhibited to at least a prescribed degree in the signals, establishing a weighted indicator whose weight represents the likelihood that the stimulus presented to the person belongs to that stimuli category;
whenever distinguishing characteristics associated with a trained stimuli category are not exhibited in the signals to at least the prescribed degree, establishing a zero weighted indicator; and
outputting the weighted indicators.

11. The process of claim 10, wherein the process action of outputting an indicator identifying the trained stimuli category that the stimulus belongs to, further comprises an action of designating that the presented stimulus belongs to the trained stimuli category associated with the largest weighted indicator.

12. The process of claim 2, wherein a stimulus has been presented to more than one person, and wherein the process further comprises the actions of:
for each person presented with a stimulus, identifying the trained stimuli category associated with the indicator that was output;
employing a voting scheme, wherein the output of an indicator identifying a particular trained stimuli category is a vote that the stimulus presented to the people belongs to that category; and
designating, based on the results of the voting scheme, which trained stimuli category that the stimulus presented to the people belongs to.

13. The process of claim 12, wherein the process action of designating which trained stimuli category that the stimulus presented to the people belongs to, comprises an action of designating that the stimulus presented to the people belongs to the trained stimuli category getting the most votes.

14. The process of claim 10, wherein a stimulus has been presented to more than one person, and wherein the process further comprises the actions of:
for each trained stimuli category, combining the weighted indicators associated with the category under consideration for all the people presented with the stimulus to produce an overall indicator for the category under consideration;
identifying which of the overall indicators has the greatest weight;
determining if the identified overall indicator exceeds a prescribed minimum weight; and
designating that the stimulus presented to the people was a stimulus belonging to the trained stimuli category associated with the identified overall indicator, whenever it is determined the identified overall indicator exceeds the prescribed minimum weight.

15. A computer-implemented process for identifying a stimuli category of a perceptual stimulus that has been presented to a person whose brain activity is being monitored, comprising using a computer to perform the following process actions:
in a training phase,
presenting to the person, at least once, a training stimulus belonging to each of one or more stimuli categories of interest,
inputting signals from a brain activity sensing device, wherein the signals exhibit different distinguishing characteristics whenever a stimulus belonging to a different one of the one or more stimuli categories is presented to the person, and wherein the distinguishing characteristics are indicative of an involuntary, subconscious response of the brain of the person to the stimulus, and
employing the inputted signals to train a detection module to recognize the respective distinguishing characteristics associated with each of the stimuli categories of interest; and
in a detection phase,
presenting a stimulus belonging to a trained stimuli category to the person a prescribed number of times,
inputting signals from the brain activity sensing device captured during a period encompassing the time the stimulus is repeatedly presented to the person and beyond,
using the detection module to identify each time distinguishing characteristics associated with a stimulus belonging to one of the trained stimuli categories are present in the inputted signals and capturing the results of the identifying,
determining which of the one or more trained stimuli categories the presented stimulus belongs to based on the captured results, and
outputting an indicator identifying the trained stimuli category that the presented stimulus belongs to.

16. The process of claim 15, wherein the process action of determining which of the one or more trained stimuli categories the presented stimulus belongs to, comprises the actions of:
for each time the stimulus was presented to the person, determining if distinguishing characteristics associated with a trained stimuli category are exhibited in the signals within a prescribed period of time following the presentation of the stimulus;
employing a voting scheme, wherein whenever it is determined that distinguishing characteristics associated with a trained stimuli category are exhibited in the signals within a prescribed period of time following the presentation of the stimulus, a vote is cast that the stimulus belongs to that stimuli category; and
designating, based on the results of the voting scheme, which of the trained stimuli categories that the stimulus presented to the person belongs to.

17. The process of claim 16, wherein the process action of designating which of the trained stimuli categories that the stimulus presented to the person belongs to, comprises an action of designating that the stimulus presented to the person belongs to the trained stimuli category getting the most votes.

18. The process of claim 15, wherein the process action of determining which of the one or more trained stimuli categories the presented stimulus belongs to, comprises the actions of:
for each time the stimulus was presented to the person,
determining the degree to which distinguishing characteristics associated with each trained stimuli category are exhibited in the signals within a prescribed period of time following the presentation of the stimulus,
whenever the distinguishing characteristics are exhibited to at least a prescribed minimum degree, establishing a weighted indicator for each trained stimuli category whose weight represents the likelihood that the stimulus presented to the person belongs to that stimuli category, and
whenever distinguishing characteristics associated with a trained stimuli category are not exhibited to at least the prescribed minimum degree, establishing a zero weighted indicator for that stimuli category;
for each trained stimuli category, combining the weighted indicators associated with the category under consideration for all the presentation instances to produce an overall indicator for the stimuli category under consideration;
identifying which of the overall indicators has the greatest weight;
determining if the identified overall indicator exceeds a prescribed minimum weight; and
designating that the stimulus presented to the person was a stimulus belonging to the trained stimuli category associated with the identified overall indicator, whenever it is determined the identified overall indicator exceeds the prescribed minimum weight.

19. The process of claim 15, wherein the process action of determining which of the one or more trained stimuli categories the presented stimulus belongs to, comprises the actions of:
for each time the stimulus was presented to the person,
determining the degree to which distinguishing characteristics associated with each trained stimuli category are exhibited in the signals within a prescribed period of time following the presentation of the stimulus,
whenever the distinguishing characteristics are exhibited to at least a prescribed minimum degree, establishing a weighted indicator for each trained stimuli category whose weight represents the likelihood that the stimulus presented to the person belongs to that stimuli category, and
whenever distinguishing characteristics associated with a trained stimuli category are not exhibited to at least the prescribed minimum degree, establishing a zero weighted indicator for that stimuli category;
for each trained stimuli category, combining the weighted indicators associated with the category under consideration for all the presentation instances to produce an overall indicator for the stimuli category under consideration;
identifying which of the overall indicators has the greatest weight;
determining if the identified overall indicator exceeds at least one of, a prescribed minimum weight, and the second largest indicator by more than a prescribed amount;
designating that the stimulus presented to the person was a stimulus belonging to the trained stimuli category associated with the identified overall indicator, whenever it is determined the identified overall indicator exceeds the prescribed minimum weight, or exceeds the second largest indicator by more than the prescribed amount, or both;
whenever it is determined the identified overall indicator does not exceed the prescribed minimum weight, or it is determined the identified overall indicator does not exceed the second largest indicator by more than a prescribed amount,
presenting the same stimulus to the person that was previously presented;
repeating the inputting, identifying and determining actions;
designating that the stimulus presented to the person was a stimulus belonging to the trained stimuli category associated with the identified overall indicator, whenever it is determined the identified overall indicator exceeds the prescribed minimum weight and exceeds the second largest indicator by more than the prescribed amount.

20. The process of claim 15, wherein the process action of presenting the stimulus belonging to the trained stimuli category to the person the prescribed number of times, comprises presenting the stimulus between one and ten times.
